(19) United States
(12) Patent Application Publication
(10) Pub. No.: US 2006/0260624 A1
(43) Pub. Date: Nov. 23, 2006

(54) Method, program, and system for automatic profiling of entities

(75) Inventors: Anne Schur, Richland, WA (US); Richard A. May, Richland, WA (US); Ryan E. Hohimer, West Richland, WA (US)

Correspondence Address: BATTELLE MEMORIAL INSTITUTE;ATTN: IP SERVICES, K1-53, P. O. BOX 999, RICHLAND, WA, 99352, US

(73) Assignee: Battelle Memorial Institute, Richland, WA (US)

(21) Appl. No.: 11/131,543

(22) Filed: May 17, 2005

(51) Int. Cl. A61B 19/00; A61B 5/00

(52) U.S. Cl. 128/898; 600/300

(57) ABSTRACT

A method for automatic profiling of an entity and a system adopting the method comprises acquiring sensory data having at least one behavioral cue of the entity; annotating the behavioral cues according to an ontology; assigning a behavioral signature to an annotated-behavioral-cue set, wherein the annotated-behavioral-cue set comprises at least one annotated behavioral cue; and associating human subjectivity information with a behavioral-signature set, wherein the behavioral-signature set comprises at least one behavioral signature.

### STATEMENT REGARDING FEDERALLY SPONSORED RESEARCH OR DEVELOPMENT

This invention was made with Government support under Contract DE-AC0576RLO1830 awarded by the U.S. Department of Energy. The Government has certain rights in the invention.

### BACKGROUND

Traditionally, observation of human behavior and/or cultural indicators is achieved by observing media such as textual descriptions, still images, audio and video. This media is then manually assessed and the findings conveyed to others in a similar manner. Examples can include broadcast monitoring, interviews, crowd observations, intelligence data collection, and speeches of leaders. The processes are time consuming, and critical patterns of behavior can be missed because a) the assessments are performed manually, b) multiple resources cannot be easily utilized in a timely manner, and c) the techniques used are influenced by the cultural bias of the observer/analyst. Thus a method for automatic profiling of entities is required to observe human behaviors and infer human subjectivity information regarding the entity, wherein human subjectivity information can include but is not limited to motives, intent, and culture.

### SUMMARY

One embodiment of the present invention encompasses a method for automatic profiling of an entity and comprises the steps of acquiring sensory data comprising at least one behavioral cue of the entity; annotating the behavioral cues according to an ontology; assigning a behavioral signature to an annotated-behavioral-cue set, wherein the annotated-behavioral-cue set comprises at least one annotated behavioral cue; and associating human subjectivity information with a behavioral-signature set, wherein the behavioral-signature set comprises at least one behavioral signature.

Another embodiment encompasses a system for automatic profiling of an entity and adopts the method described above. The system comprises a program on a computer readable medium having logic to handle sensory data, wherein the sensory data comprises at least one behavioral cue The program further comprises logic to annotate the behavioral cues according to an ontology; logic to assign a behavioral signature to an annotated-behavioral-cue set comprising at least one annotated behavioral cue; and logic to associate human subjectivity information with a behavioral-signature set, wherein the behavioral-signature set comprises at least one behavioral signature. The system also comprises at least one instrument for collecting the sensory data and at least one device for implementing the program.

### DESCRIPTION OF DRAWINGS

Embodiments of the invention are described below with reference to the following accompanying drawings.

FIG. 1 is a flow chart illustrating an embodiment of a method for automatic profiling.

FIG. 2 depicts an example of a wire diagram of a human hand.

FIG. 3 is a flowchart of an embodiment for an artificial neural network.

FIG. 4 is a flowchart of an embodiment for an artificial neural network.

### DETAILED DESCRIPTION

For a clear and concise understanding of the specification and claims, including the scope given to such terms, the following definitions are provided.

An entity, as used herein, can refer to an individual, a group of individuals, and/or a society of interest.

Behavioral cues can comprise any observable behaviors including, but not limited to, head direction, gaze direction, body posture, facial expressions, gestures, word-sense usage, prosody, speech disfluencies, physiological responses, and combinations thereof. Word-sense can refer to the accepted meaning of a particular word or phrase. Often, the accepted definition of a word in one culture is quite different than the same word in another culture. Prosody can refer to the pitch, stress, timing, loudness, tempo, accent, and/or rhythm patterns of spoken language that result in phrasing and expression. As with word sense, prosody can vary among cultures, sub-cultures, and even individuals. Speech disfluencies typically occur in spontaneous speech and can comprise pauses, elongated segments, repetitions, stammering, self-corrections, false starts, and filled pauses (e.g., saying, “uh,” “um,” etc.).

Human subjectivity information, as used herein, can refer to information and/or characterizations relating behavioral signature sets with the subjective aspects of human behaviors and mental processes. These subjective aspects can include, but are not limited to, culture, intent, motives, emotions, conditioned responses, and learning styles. Thus, human subjectivity information can comprise information relating behavioral signatures to cognitive states, emotional states, motivations, cognitive processes, cultural characteristics, specific behavioral sequences, procedures of behavior, beliefs, values, and combinations thereof.

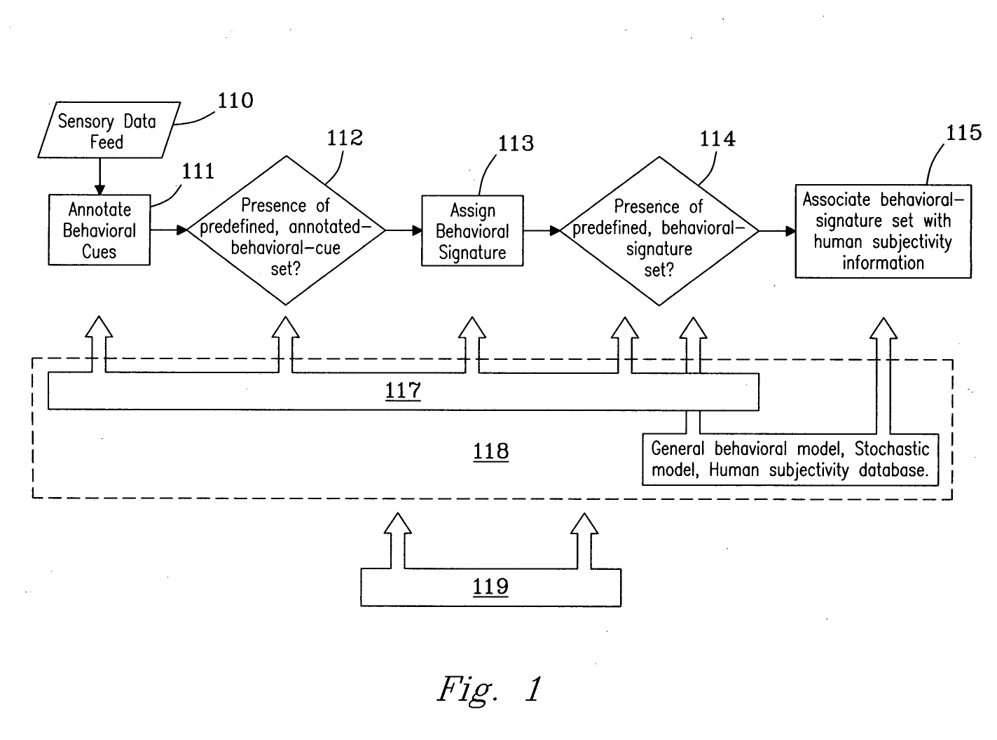

Referring to the embodiment represented in FIG. 1, sensory data from an entity of interest is processed 110 to annotate any behavioral cues 111 that might be present in the data. Annotation of the behavioral cues is accomplished according to an ontology 117. Based on definitions in the ontology, annotated-behavioral-cue sets comprising at least one behavioral cue can be identified. If an, annotated-behavioral-cue set is present 112 among the sensory data, the set is assigned a behavioral signature 113. In a manner similar to the handling of behavioral cues, arrangements of at least one behavioral signature can be classified into a behavioral-signature set. The behavioral signature sets can be determined according to a cultural data warehouse 118 comprising the ontology, a general behavioral model, a stochastic model, and/or a human subjectivity information database. If a predefined, behavioral-signature set is found 114, the behavioral-signature set can be associated with human subjectivity information 115. Furthermore, the cultural data warehouse 118 can be updated according to input and feedback from a user 119. Such associations result in the automatic profiling of the entity and its behavior.

In another embodiment, a system for automatic profiling comprises a program on a computer readable medium, a human cultural data warehouse, and at least one instrument for collecting sensory data. The program can comprise logic to handle the sensory data, to annotate the behavioral cues, to assign a behavioral signature to an annotated-behavioral-cue set comprising at least one annotated behavioral cue, and to associate human subjectivity information with at least one behavioral-signature set. The human cultural data warehouse can comprise the ontology, the general behavior model, and/or the human subjectivity information database.

Instantiation of behavioral cues involves acquiring sensory data and annotating behavioral cues from an entity of interest. Sensory data can comprise information provided by sensor devices, which include, but are not limited to, video sensors, audio sensors, piezo-resistive flex sensors, electro-optical sensors, electromagnetic sensors, biometric sensors, thermometers, pressure sensors, and accelerometers. In one embodiment, annotation can be achieved according to a predefined ontology, which can be accessed to apply a label to a behavioral cue. For example, in a sensory data video stream, an entity's behavioral cue of touching an ear can be labeled as such, based on an ontology that relates the placement of a hand on an ear as an “ear touch.” In the example, the label can be textual for subsequent processing by a natural language processing engine, as will be described below. In another instance, the label can be graphical, symbolic, numerical, and/or color-based.

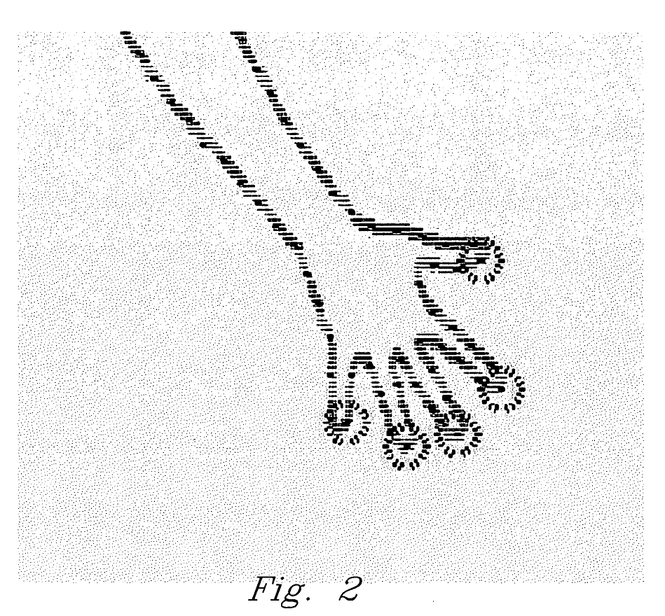

An example of a graphical label, referring to FIG. 2, can include configurations of a human hand labeled as a geometric shape by representing the hand configurations with a wire diagram. Another example of a graphical label can include a data graph and/or a spectrograph. The use of non-textual labels can allow for utilization of multimodal sensory data and can minimize the redefining of the significance of a textual word. For example, the terms “we,” “us,” and “ours,” which can compose behavioral cues in a text document to signify a group of individuals, would not serve as effective labels for behavioral cues in a video stream. Instead, for example, colors can be used to denote individuals belonging to subgroups. Thus, annotations of behavioral cues can comprise abstract representations.

A number of sensor devices are included in commercially-available movement tracking systems, the use of such devices, and those with substantially similar function, is encompassed by embodiments of the present invention. One such system is the MOTIONSTAR™ manufactured by Ascension Technologies (Burlington, Vt.). Another example can include a video sensor, such as a camera, to collect pictures and/or video containing an individual's gaze direction, body posture, and/or hand gestures. Details regarding an example of using video are provided by Meservy et al., which details are incorporated herein by reference (Meservy et al., Automatic Extraction of Deceptive Behavioral Cues from Video, Proceedings from IEEE International Conference on Intelligence and Security Informatics, Intelligence and Security Informatics, Atlanta, Ga., USA, (2005) pg. 198-208). Additionally, infrared video sensors can detect body temperature, while pressure sensors can monitor gait properties. Yet another example is an audio sensor that can collect a recording of an individual's speech, which might exhibit prosodic features and/or speech disfluencies. Alternatively, sensory data can comprise information provided by information tools, which include, but are not limited to, surveys, questionnaires, census data, information databases, transcripts of spoken words, specific or ad-hoc writing samples, historical records, and combinations thereof.

As alluded to above, some embodiments of the invention can utilize a combination of sensor-device types in order to acquire multimodal sensory data. Any combination can be acceptable (e.g., audio, biometric, two video sensors, and a written survey), and the suitability of particular sensor-device/information tool configurations will be dictated by each application. While a number of sensor device technologies for collecting sensory data exist, and have been described, the technologies provided herein are exemplary and are not intended to be limitations of the invention.

Annotation of behavioral cues can be accomplished according to an ontology. As detailed by Thomas Gruber in Knowledge Acquisition (Vol. 5, Issue 2, June 1993, Pages 199-220), which details are incorporated herein by reference, an ontology can refer to “an explicit specification of a conceptualization,” wherein a conceptualization includes objects, concepts, events, and other features that are presumed to exist in an area of interest. Thus, the ontology can provide a representation of behavioral cues that is applied to identify the cues from the sensory data. For example, an ontology might define the word “um” as a behavioral cue, wherein “um” is uttered by individuals during pauses in speech. It can further describe relationships between “um” and a number of languages in which the utterance is common, for example, American English, British English, French, and Spanish. An embodiment of the present invention, established to monitor people at an airport, might record a conversation between two German-speaking individuals. During the course of the conversation, the first individual might say “um” during pauses in speech. Sensory data (audio) comprising a transcript of the conversation can be fed to a device for processing and identification of behavioral cues. The device would identify the presence of the “um” utterances and annotate them for analysis according to the provided ontology. In conjunction with a number of other annotated behavioral cues, which can compose a behavioral signature, the instant embodiment might characterize the first individual as an American that has learned to speak German fluently, as opposed to a native German speaker.

The elements in an ontology can be defined by the user, wherein the user specifies annotations for specific behavioral cues. Similarly, the ontology can be provided by models. In one embodiment, the ontology can include parametrics from behavioral correlates of deception and/or gesture cues. Details regarding these parametrics are described in Burgoon, J. K., Buller D. B. Interpersonal deception: III. Effects of deceit on perceived communication and nonverbal behavior dynamics. Journal of Nonverbal Behavior, 18, 155-184 (1994); and DePaulo et al. Cues to Deception. Psychological Bulletin, 129(1), 74-118 (2003); which details are herein incorporated by reference. Additional parameterics can be derived from neuro-linguistic programming (NLP). Alternatively, NLP concepts and models themselves can provide the basis for the ontology. Another example of a model includes, but is not limited to, Hofstede's Cultural Dimension. The use of existing ontologies, such as WordNet®, Web Ontology Language (OWL), Video Event Representation Language (VERL), and Video Event Markup Langauge (VEML) is also encompassed by embodiments of the present invention. WordNet® is an online lexical reference system whose design was inspired by psycholinguistic theories of human lexical memory. English nouns, verbs, adjectives and adverbs are organized into synonym sets, each representing one underlying lexical concept. Different relations link the synonym sets.

One embodiment, which serves as an example of acquiring sensory data and annotating behavioral cues, utilizes speech and natural language processing technologies. Speech and processing technologies can include, but are not limited to, automatic speech recognition tools such as Dragon NaturallySpeaking™ (ScanSoft®, Peabody, Mass.). An information extraction technology like the General Architecture Text Engineering (GATE, http://gate.ac.uk) can be informed by an ontology, to distill relevant linguistic patterns from transcriptions provided by Dragon NaturallySpeaking™ from audio-based sensory data. Thus, audio sensors, in addition to the speech recognition software, can generate text-based sensory data. The sensory data is processed by GATE, which uses the ontology to annotate behavioral cues. When an annotated-behavioral-cue set, comprising at least one annotated behavioral cue, is identified for having a known pattern of behavioral cues, a behavioral signature is assigned to the annotated-behavioral-cue set. In some embodiments, the annotated behavioral cues are processed using natural language processing tools to recognize patterns. Assignment of the behavioral signature can occur based on an ontology as in this example, or it can occur based on a general behavior model, which is described below in further detail.

In another embodiment, prosodic features can be annotated according to speech analysis software including, but not limited to the Summer Insitute of Learning's (SIL) Speech Analyzer. (http://www.sil.org/computing/speechtools) and Praat (http://www.fon.hum.uva.nl/praat), which was developed by the Institute of Phonetic Sciences at the University of Amsterdam.

Behavioral signatures comprise at least one behavioral cue and can be suggestive of characteristics unique to an entity of interest. The significance of the behavioral signatures can be interpreted according to the context in which the entity exists and/or the particular application. Applications can include, but are not limited to cultural characterization, educational-technique enhancement, consumer business modeling, human-behavior prediction, human-intent detection, criminal profiling, intelligence gathering, crowd observation, national security threat detection, and combinations thereof.

Behavioral signatures can be assigned according to characterizations including those provided, but not limited by, cognitive models, cultures, sub-cultures, processing style, and combinations thereof. They can also be based on repetition of one or more behavioral cues. For example, an entity's behavior, which alone may have no apparent cognitive or cultural significance, when exhibited repeatedly in a relatively short period of time can be utilized as effective indicia. Similarly, context can provide a basis for assigning significance to a behavioral signature. A heavily-perspiring individual might generate greater scrutiny in an air-conditioned airport than in a gymnasium.

In one embodiment, behavioral signatures can be predetermined and included in an ontology and/or a general behavior model. As used herein, a general behavior model is distinguished over an ontology by the level of relationships defined therein. Whereas an ontology can define and link fundamental elements, general behavior models define and link complex elements and/or a plurality of fundamental elements. For example, an ontology might be used to deterministically annotate basic behavioral cues and assign a behavioral signature to a set of the annotated behavioral cues. In contrast, the general behavior model would be used to associate a plurality of behavioral signatures with human subjectivity information. Such association can be deterministic and/or stochastic.

A general behavior model can include, but is not limited to, stereotypes and/or models of the entity's characteristics based on culture, cognitive process, gender, social class, personality, physical characteristics, habits, historical behavior, and combinations thereof. Specific general behavior models can comprise neuro-linguistic programming models, model-based assessments, communication hierarchies, Hofestede's cultural dimensions, cognitive-situation assessment models, and combinations thereof.

In another embodiment, the stochastic assigning and associating of behavioral signatures can occur opportunistically according to an artificial neural network (ANN). Artificial neural networks (ANN) can be used to categorize, organize, and detect patterns in detected behavioral cues and signatures. The input for the ANN can comprise sensory data, behavioral cues, behavioral-cue sets, behavioral signatures, and/or behavioral signature sets. In one example, the input layer can comprise N feeds from the sensor devices and/or information tools and an output layer represents a range of N possible detected higher level events.

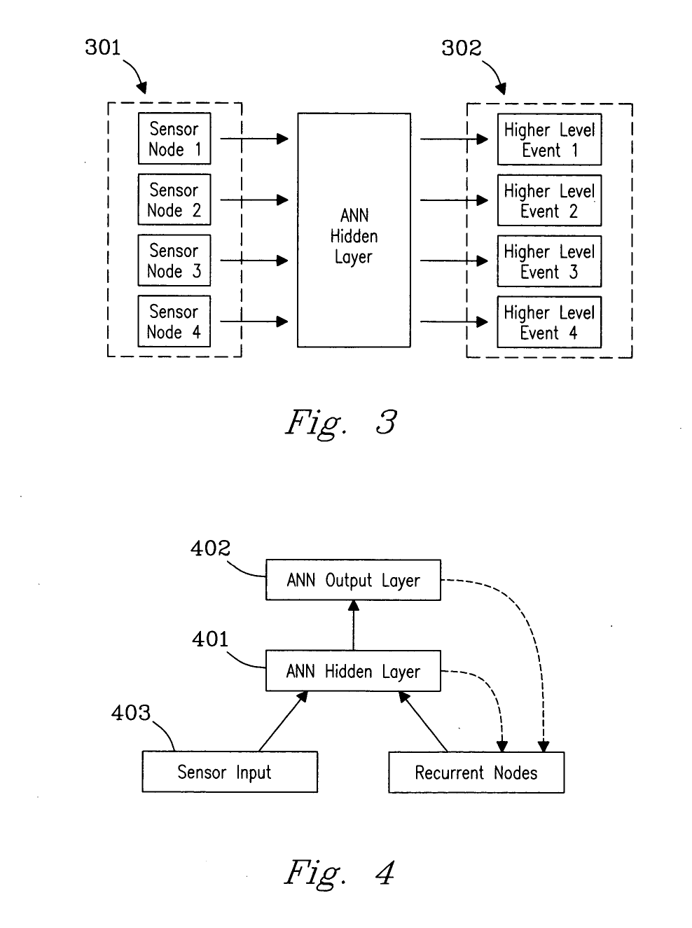

An example of an ANN design is depicted in FIG. 3. The sensor input nodes 301 can represent a series of N detected events over time. The number of input nodes would be bounded by the total number of input events needed to capture all possible desired output events. This is a common approach to dealing with time varying input to an ANN. The ANN output layer 302 can comprise at least one high-level event node node. The end result is the same except that the value of the single output node determines which event has been detected. In order to allow greater hierarchies of detected events, hierarchies of networks can be developed wherein the output of one network serves as the input or partial input to another network.

FIG. 4 shows another example of an ANN comprising a recurrent ANN. A traditional ANN can be referred to as a feed-forward ANN because all information travels in only one direction—from input to output. In a recurrent ANN the results of either the hidden layer 401 or the output layer 402 can be used as additional input. So whenever new input is received by the network it can be combined with hidden or output node results from the previous times the ANN was run. When using a recurrent ANN only one sensor input 403 would be used at a time. Because each sensor detected event is combined with previous event information by the recurrent feeds there is a maintained knowledge base of previous sensor detected events. The recurrent ANN can also inherently support hierarchical events. In one embodiment, several recurrent ANNs can be used to help classify behavioral cues.

Training of the ANN can occur regardless of the type of ANN used. One common approach to training comprises pre-training the ANN based on known inputs with known results. The traditional algorithm is the back-propagation technique, which is encompassed by embodiments of the present invention. Another approach comprises dynamically training the ANN, which can involve training the ANN during use. Thus, performance of the ANN can improve over time.

Assignments and/or associations of behavioral signatures can be refined according to observed patterns and/or input from users or administrators. The input provided by the user can include, but is not limited to, actions selected from the group consisting of adding to the ontology, defining behavioral signatures, confirming assignments, confirming associations, rejecting assignments, rejecting associations, and combinations thereof. Accordingly, a behavioral signature can comprise an aggregation of behavioral cues according to a pattern. Some embodiments can comprise a pattern-recognition algorithm to aggregate the behavioral cues according to a set of rules. Alternatively, behavioral cues can be displayed visually using data visualization software. Based on the displayed data, users can recognize patterns and confirm or reject an automatic assignment. In so doing, future assignments can be made with a greater confidence value.

In one example, a pattern can include repeated observation of reading and spelling errors such as letter reversals (b/d), letter inversions (m/w), and/or transpositions (felt/left). The presence of a repetition of these errors (i.e., behavioral cues) can be defined as a behavioral signature, which can ultimately be characteristic of a learning disability in the entity associated with the errors. Behavioral signatures defined according to observed patterns of behavioral cues do not need to be predefined and can, therefore, be generated and/or updated in real-time as behavioral cues are identified and aggregated according to such observed patterns. Aggregation can occur opportunistically without human intervention and/or from specific patterns defined by a user (e.g., analyst or researcher).

According to embodiments of the present invention, human subjectivity information, which can be stored in a database, is associated with behavioral-signature sets comprising at least one behavioral signature. Associations can be made in a similar manner as described earlier for the assigning of behavioral signatures. Specifically, associations can be made deterministically based on an ontology and/or general behavior model. They can also be made opportunistically based on, for example, an ANN. The association can suggest a significance and/or interpretation of the behavioral-signature set. For example, some behaviors are commonly observed in one culture, but not in others. Therefore, the human subjectivity database, which can contain information relating certain behavioral-signature sets with particular cultures, can be accessed to provide meaning for a given behavioral-signature set. If the behavioral-signature set matches behaviors associated with the particular culture, it can suggest that the entity associated with the behavioral signature might have had significant exposure to that culture. A similar analysis can be performed using other human subjectivity information and would fall within the scope of the present invention.

While a number of embodiments of the present invention have been shown and described, it will be apparent to those skilled in the art that many changes and modifications may be made without departing from the invention in its broader aspects. The appended claims, therefore, are intended to cover all such changes and modifications as they fall within the true spirit and scope of the invention.

We claim:

1. A method for automatic profiling of an entity comprising the steps of:
a. acquiring sensory data comprising at least one behavioral cue of the entity;
b. annotating the behavioral cues according to an ontology;
c. assigning a behavioral signature to an annotated-behavioral-cue set, wherein the annotated-behavioral-cue set comprises at least one annotated behavioral cue; and
d. associating human subjectivity information with a behavioral-signature set, wherein the behavioral-signature set comprises at least one behavioral signature.

2. The method as recited in claim 1, wherein said annotating comprises labeling behavioral cues with text, graphics, symbols, numbers, colors, data graphs, spectrographs, diagrams, or combinations thereof.

3. The method as recited in claim 1, wherein the sensory data comprises multimodal sensory data.

4. The method as recited in claim 1, wherein the sensory data comprises information provided by sensor devices.

5. The method as recited in claim 4, wherein the sensor devices are selected from the group consisting of video sensors, audio sensors, biometric sensors, thermometers, accelerometers, or combinations thereof.

6. The method as recited in claim 1, wherein the sensory data comprises information provided by an information tool.

7. The method as recited in claim 6, wherein the information tool is selected from the group consisting of surveys, questionnaires, census data, information databases, historical records, and combinations thereof.

8. The method as recited in claim 1, wherein the automatic profiling occurs substantially in real-time.

9. The method as recited in claim 1, wherein the behavioral cues comprise behaviors selected from the group consisting of gaze direction, word sense categories, speech disfluencies, prosodic features, body posture, facial expressions, gestures, and combinations thereof.

10. The method as recited in claim 1, wherein the entity is an individual, a group, or a society.

11. The method as recited in claim 1, wherein the assigning step, the associating step, or a combination thereof are accomplished using an artificial neural network.

12. The method as recited in claim 1, wherein the assigning step further comprises recognizing patterns of annotated behavioral cues.

13. The method as recited in claim 12, further comprising the step of representing the patterns visually.

14. The method as recited in claim 12, wherein the recognizing is deterministic or stochastic.

15. The method as recited in claim 14, wherein the deterministic recognizing is based on an ontology.

16. The method as recited in claim 15, wherein the ontology comprises a model selected from the group consisting of parametrics, derivations of neuro-linguistic programming concepts, model-based assessments, communication hierarchy, Hofestede's cultural dimensions, cognitive situation assessment models, and combinations thereof.

17. The method as recited in claim 1, the associating step further comprises comparing the behavioral-signature set with a human subjectivity database.

18. The method as recited in claim 17, wherein the human subjectivity database comprises data relating behavioral signatures to cognitive states, emotional states, motivations, cognitive processes, cultural signatures, or combinations thereof.

19. The method as recited in claim 17, further comprising the step of refining the human subjectivity database with the behavioral-signature set.

20. The method as recited in claim 17, further comprising the step of predicting future behavior of the entity from the behavioral-signature set.

21. The method as recited in claim 1, further comprising the step of updating a cultural data warehouse according to input from a user.

22. The method as recited in claim 21, wherein the input from the user is selected from the group consisting of adding to the ontology, defining behavioral signatures, confirming assignments, confirming associations, rejecting assignments, rejecting associations, and combinations thereof.

23. The method as recited in claim 1, wherein the automatic profiling comprises characterizing a cognitive state, emotional state, motivation, cognitive process, intent, cultural signature, or combination thereof of the entity based on behavioral cues.

24. The method as recited in claim 1, implemented on a computer.

25. A system comprising:
a. at least one instrument for collecting sensory data, said sensory data comprising behavioral cues;
b. a computer readable medium comprising a program, said program comprising:
i. logic to handle said sensory data, wherein the sensory data comprises behavioral cues;
ii. logic to annotate the behavioral cues according to an ontology;
iii. logic to assign a behavioral signature to an annotated-behavioral-cue set comprising at least one annotated behavioral cue;
iv. logic to associate human subjectivity information with a behavioral-signature set, wherein the behavioral-signature set comprises at least one behavioral signature; and
c. at least one device for implementing the computer readable medium;
wherein said system enables automatic behavior detection and profiling of at least one entity.

26. The system as recited in claim 25, wherein the instrument comprises a device selected from the group consisting of video sensors, audio sensors, biometric sensors, thermometers, accelerometers, surveys, census data, writing samples, and combinations thereof.

27. The system as recited in claim 25, wherein said behavioral cues comprise behaviors selected from the group consisting of gaze direction, word sense categories, speech disfluencies, prosodic features, body posture, facial expressions, gestures, etc., and combinations thereof.

28. The system as recited in claim 25, further comprising logic to enable a user to update a cultural data warehouse.

29. The system as recited in claim 25, wherein said computer readable medium further comprises a human subjectivity database

30. The system as recited in claim 25, further comprising logic to predict future behavior of the entity based on the behavioral-signature set.

31. The system as recited in claim 25, further comprising a learning algorithm.

32. The system as recited in claim 31, wherein the learning algorithm expands the human subjectivity database with behavioral-signature sets.

33. The system as recited in claim 25, wherein said automatic profiling of said entity comprises characterization of cognitive states, emotional states, motivations, cognitive processes, cultural signatures, or combinations thereof based on said behavioral cues.
